(19) United States
(12) Patent Application Publication
(10) Pub. No.: US 2016/0150995 A1
(43) Pub. Date: Jun. 2, 2016

(54) PORTABLE DEVICE FOR PERSONAL BREATH QUALITY AND DEHYDRATION MONITORING

(71) Applicant: Breathometer, Inc., Burlingame, CA (US)

(72) Inventors: Tim Ratto, Milbrae, CA (US); Kenton Ngo, San Francisco, CA (US); Jonathan Gallagher, San Francisco, CA (US); Likang Xue, Santa Clara, CA (US); Silpesh Patel, Brisbane, CA (US)

(21) Appl. No.: 14/943,803

(22) Filed: Nov. 17, 2015

Related U.S. Application Data
(60) Provisional application No. 62/085,432, filed on Nov. 28, 2014.

(51) Int. Cl. A61B 5/08; A61B 5/00

(52) U.S. Cl. CPC A61B 5/082; A61B 5/0022; A61B 5/7282; A61B 5/7225; A61B 2562/029

(57) ABSTRACT

Disclosed is a breath quality analysis device having a housing, a sensor, and a mouthpiece. The housing includes an inlet opening, one or more outlet openings, and an inner cavity. The inner cavity defines a path between the inlet opening and the one or more outlet openings. The sensor is disposed inside the inner cavity of the housing. The mouthpiece is coupled to the inlet opening of the housing and includes a blower configured to draw air into the inner cavity of the housing.

### CROSS REFERENCE TO RELATED APPLICATIONS

This application claims the benefit of U.S. Provisional Application No. 62/085,432, filed Nov. 28, 2014, which is incorporated by reference in its entirety.

### BACKGROUND

1. Field of the Disclosure

The present disclosure relates to an oral analysis device and more specifically to a portable breath quality and dehydration monitoring device.

2. Description of the Related Art

Poor breath quality may be caused by multiple factors such as food, poor hygiene, and dehydration. For instance, certain foods such as garlic or onion may cause bad breath in a person. In addition, certain disorders such as gum disease, bacteria and fungi growth in a person's tongue and dehydration may cause bad breath. Eating mints or chewing gum may disguise the bad breath but without treating the root cause of the bad breath, the improved oral quality is only temporary.

Furthermore, breath contains multiple biomarkers that may be used to detect or diagnose multiple health disorders. For instance, biomarkers in breath can be used for glucose monitoring or lung cancer detection. The detection and measurements of such biomarkers are commonly performed by laboratory grade instrument, such as a halimeter.

### SUMMARY

Embodiments of the present disclosure provide a breath quality analysis device. The breath quality analysis device determines an oral quality of a user by measuring certain analytes present in the breath of the user. The analytes analyzed includes volatile sulfur compounds VSC such as methyl mercaptan (CH₄S), hydrogen sulfide (H₂S), and dimethyl sulfide ((CH₃)₂S). in addition, the breath quality analysis device may determine the relative humidity of the breath sample to determine a hydration level of the user.

The breath quality analysis device includes a housing, a sensor, and a mouthpiece. The housing includes an inlet opening, one or more outlet openings, and an inner cavity. The inner cavity defines a path between the inlet opening and the one or more outlet openings. The sensor is disposed inside the inner cavity of the housing. The mouthpiece is coupled to the inlet opening of the housing and includes a blower configured to draw air into the inner cavity of the housing.

### BRIEF DESCRIPTION OF DRAWINGS

The disclosed embodiments have other advantages and features which will be more readily apparent from the detailed description, the appended claims, and the accompanying figures (or drawings). A brief introduction of the figures is below.

FIG. 1 illustrates the operating architecture of a breath quality and dehydration monitoring system for analyzing volatile sulfur compounds (VSCs) and dehydration of a user, according to one embodiment.

FIG. 2A illustrates a top view of a breath analysis device, according to one embodiment.

FIG. 2B illustrates a cross-sectional side view of the breath analysis device, according to one embodiment.

FIG. 2C is a top view of the mouthpiece of the breath analysis device, according to one embodiment

FIG. 2D is a cross-sectional side view of the mouthpiece of the breath analysis device, according to one embodiment.

FIG. 3A illustrates a sample breath flow inside the breath analysis device, according to one embodiment.

FIG. 3B illustrates a box diagram of a sample breath flow inside the breath analysis device, according to one embodiment.

FIG. 4 illustrates a flow diagram of a process for testing the breath quality of a user, according to one embodiment.

FIG. 5 illustrates a graph of the output of the sensor 235 during an exemplary breath quality test, according to one embodiment.

FIG. 6 illustrates a flow diagram of a process for determining a VSC concentration in a user's breath, according to one embodiment.

FIG. 7 illustrates an offset response and a sample response after being adjusted for a baseline response (V_{begin}) of a sensor exposed to a sample with a high RH and with a temperature of 37° C., according to one embodiment.

FIG. 8 illustrates a user interface for controlling the breath analysis device, according to one embodiment.

FIG. 9 illustrates a user interface for providing instructions to a user during the analysis of the user's breath, according to one embodiment.

FIG. 10A illustrates a user interface for providing the analysis results, according to one embodiment.

FIG. 10B illustrates a user interface for providing the analysis results, according to another embodiment.

FIG. 11 illustrates a user interface to display data from previous test performed by the breath analysis device, according to one embodiment.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Breath Analysis Operating Architecture

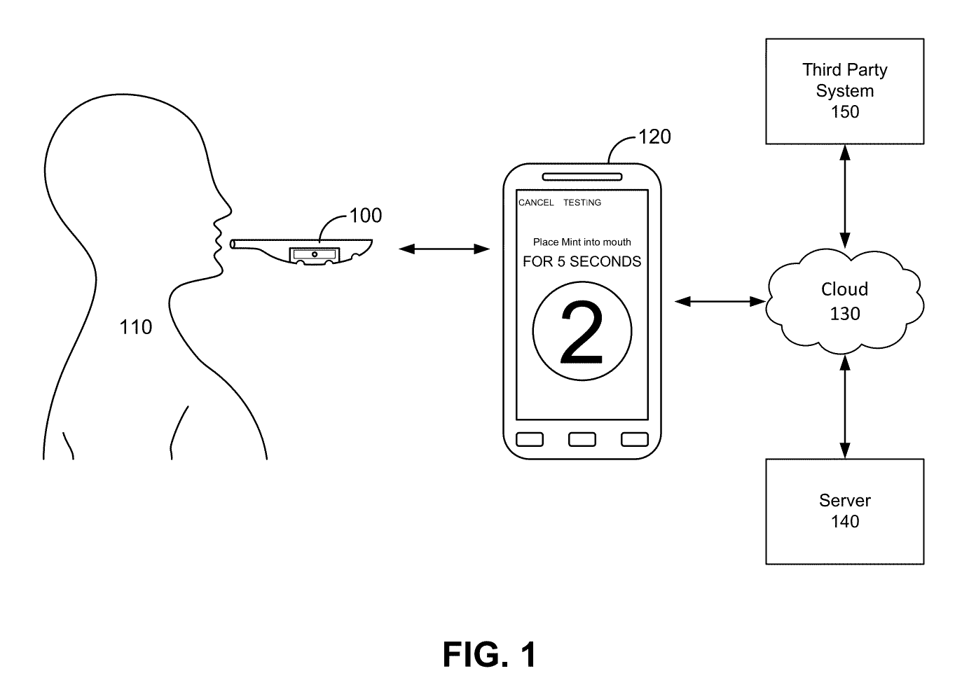

FIG. 1 illustrates an operating architecture of a breath analysis system for analyzing different compounds, such as volatile sulfur compounds (VSCs), in the breath of a user, according to one embodiment. The breath analysis system includes a breath analysis device 100, a user 110 using the breath analysis device 100, and a client device 120 connected to the breath analysis device 100. In some embodiments, the client device 120 is a handheld computing device, such as a smartphone. The client device 120 may connect to the breath analysis device 100 via a wired connection or wirelessly (e.g., via Bluetooth). The breath analysis device 100 measures the concentration of chemicals in the mouth that are indicative of poor-smelling breath and/or poor oral hygiene. In particular, to measure oral hygiene rather than components of other parts of a user's breath or lung air, the breath analysis device 100 draws breath from the user's mouth without requiring the user to expressly exhale into the breath analysis device 100.

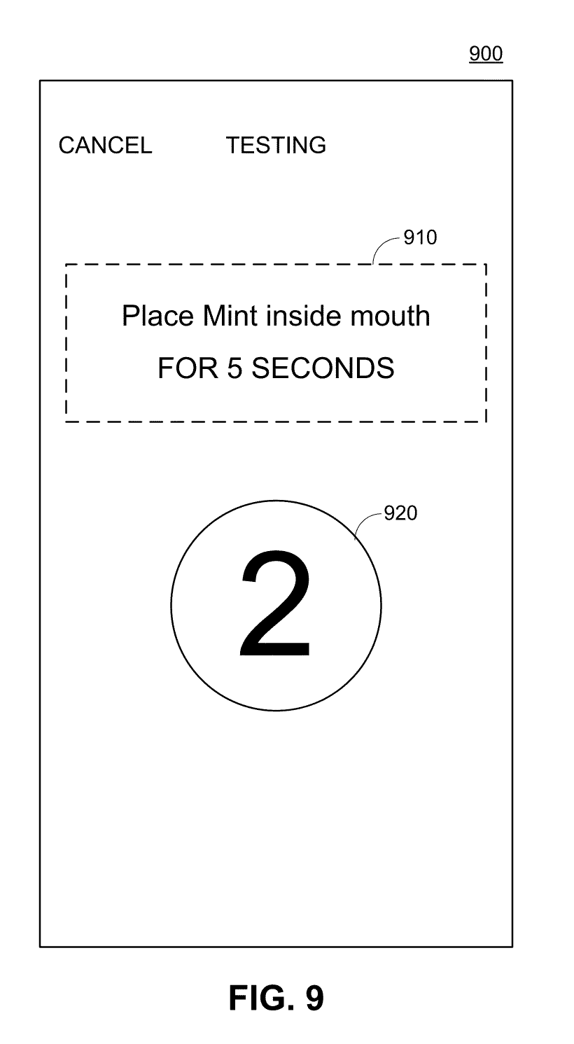

The client device 120 may receive an indication from the user to start the analysis. In some embodiments, the client device 120 provides instructions to the user 110 for performing the analysis with the breath analysis device 100. For instance, the client device 120 may instruct the user to place the breath analysis device inside the user's mouth for a predetermined amount of time (e.g. 5 seconds). The client device 120 may additionally display a countdown of the number of seconds left to complete the analysis. The client device 120 may also initialize the breath analysis device 100 prior to instructing the user. A more detailed description of the user interface displayed by the client device is provided in conjunction with FIG. 9.

The client device 120 may be configured to communicate via the network 130, which may comprise any combination of local area and/or wide area networks, using both wired and/or wireless communication systems. In one embodiment, the network 130 uses standard communications technologies and/or protocols. For example, the network 130 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 130 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 120 may be represented using any suitable format, such as hypertext markup language (HTML) or extensible markup language (XML). In some embodiments, all or some of the communication links of the network 130 may be encrypted using any suitable technique or techniques.

In some embodiments, the client device 120 communicates with a server 140 via network 130. The server may store results of the breath analysis from several devices and keep track of the performance of the breath analysis devices and may recalibrate the breath analysis devices 100 periodically. The server may also associate certain devices with specific manufacturing conditions, such as a specific lot, manufacturing version, or timeframe. The server may keep track of a drift in the measurements of a user's breath for breath analysis devices of a specific lot to identify that the devices associated with the lot need recalibration. The server identifies client devices 120 connected to a breath analysis device 100 associated with the specific lot and sends an update of calibration parameters. The calibration parameters may be applied by the mobile device 120 or may update settings on the breath analysis device 100. In some embodiments, the server updates the settings when a shift in the results of the analysis is larger than a threshold value. In other embodiments, the server sends the update periodically (e.g., every 6 months).

One or more third party systems 150 may be coupled to the network 130 for communicating with the client device 120 and/or the server 140. In some embodiments, the third party system 150 is an oral health care service provider. For instance, the third party system 150 communicates with the user's dentist and/or dental insurance provider. As such, the user's dentist may be able to receive information regarding the user's daily oral hygiene.

### Sampling for Breath Analysis Devices

Measurements that use breath to assess oral hygiene, such as volatile sulfur chemicals (VSCs) measurements, use air sampled from the mouth instead of from the lungs of the user. Typically, the detection of VSCs in a user's breath is performed using large and expensive instruments, such as Halimeters, particularly, when detecting low concentrations of VSCs (e.g., 50 to 500 parts per billion (ppb)). Additionally, these devices typically to draw air from the user's mouth instead of the user's lungs, a pump is used to obviate the user from having to blow into the analysis device to push air for the analysis. All these components increase the size, price, as well as the power consumption of these breath analysis instruments.

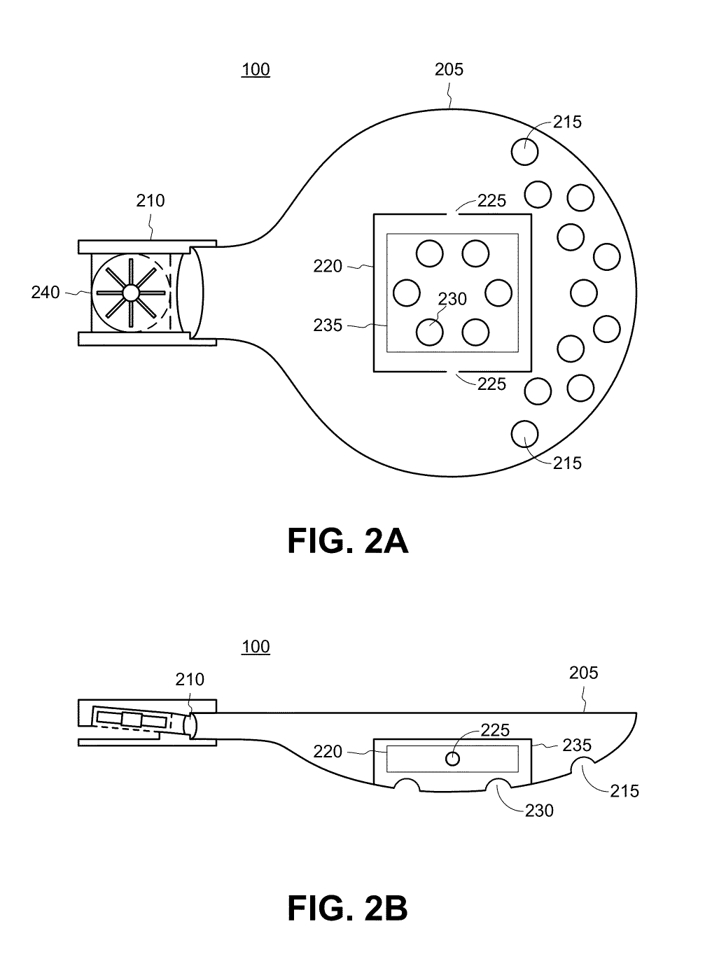

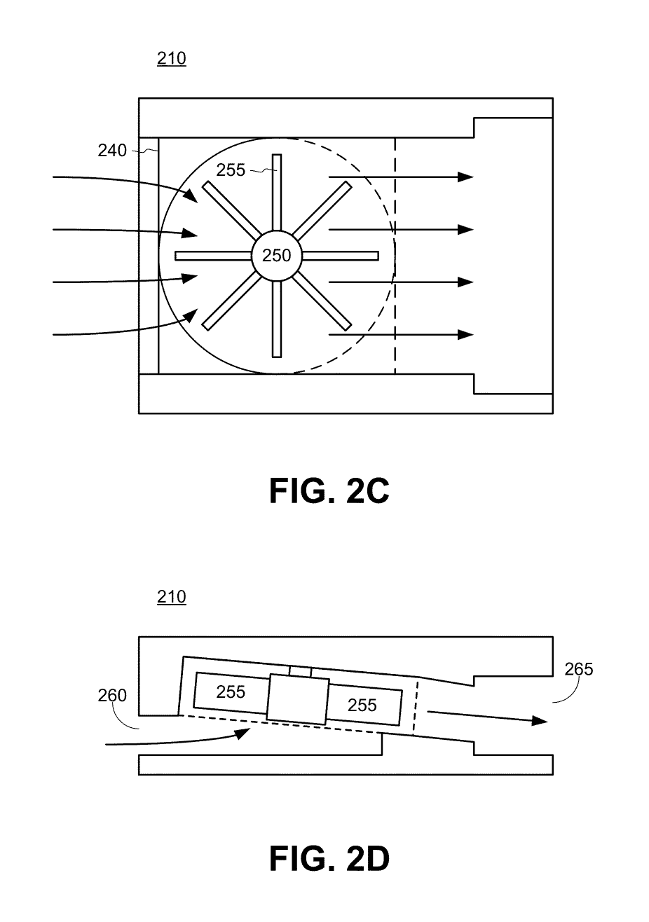

FIG. 2A illustrates a top view of a breath analysis device and FIG. 2B illustrates a cross-sectional side view of the breath analysis device, according to one embodiment. The breath analysis device 100 permits effective sampling of breath using a handheld device that draws air from the user's mouth. The breath analysis device 100 includes a housing 205, a mouthpiece 210, a blower 240 included inside the mouthpiece 210, one or more outlet holes 215, a sensor housing 220, one or more sensor inlet holes 225, one or more sensor outlet holes 230, and a sensor 235. The breath analysis device may include additional components such as a battery, a controller module, and/or a wireless transceiver.

The housing 205 includes an inner cavity that houses the internal components of the breath analysis device. At one end of the housing 205, the breath analysis device includes a mouthpiece 210. The mouthpiece 210 includes an opening to receive a breath sample from a user. In some embodiments, the mouthpiece 210 is an attachment to the housing 205. In other embodiments, the mouthpiece 210 is part of the housing 205.

FIG. 2C is a top view of the mouthpiece 210 and FIG. 2D is a cross-sectional side view of the mouthpiece 210. The mouthpiece includes an inlet 260 and an outlet 265. The mouthpiece 210 also includes a blower 240. The blower 240 draws air from the mouthpiece inlet 260 and pushes the drawn air through the mouthpiece outlet 265. The blower 240 includes a rotor 250 having multiple blades 255. In the example of FIG. 2C and FIG. 2D, blades 255 are arranged vertically. In other examples, the blades may be slanted or curved with respect to the axis of the rotor 250. As the blades 255 rotate, blades 255 push air forward into the mouthpiece outlet 265. As the air is pushed forward, air is drawn from the mouthpiece inlet 260. When the mouthpiece inlet 260 is placed inside the user's mouth, the blower 240 draws air from inside of the user's mouth into the inner cavity of the housing 205.

In some embodiments, the mouthpiece 210 includes grooves to guide the placement of the mouthpiece inside the user's mouth. For instance, in one embodiment the mouthpiece includes bite marks. The user using the breath analysis device may bite into the bite marks to guide the placement of the mouth piece of the breath analysis device inside the user's mouth. In some embodiments, the grooves are in the upper portion of the mouthpiece 210 to guide the placement of the breath analysis device with respect to the maxilla. The maxilla of a human is fixed to the skull and thus, guiding the placement of the mouthpiece based on the maxilla, as opposed to the placement of the mouthpiece based on the mandible increases the consistency of the placement of the mouthpiece.

In this embodiment, as the breath sample enters the housing 205, some of the breath sample enters a sensor housing 220 and some of the breath sample exits the housing 205 without entering the sensor housing 220. Referring back to FIG. 2A and FIG. 2B, the housing 205 additionally includes one or more outlet holes 215 to release the breath sample that does not enter the sensor housing 220. The outlet holes 215 may be 0.5 mm to 5 mm in diameter. The housing 205 may include 1 to 50 outlet holes 215. In some embodiments, the number and size of the outlet holes 215 are based on the amount of air to be released from the breath analysis device. For instance, the size and number of outlets holes 215 may be proportional to the size of the opening of the mouthpiece 210. In particular, the number and size of outlet holes 215 may vary the resistance of air to leaving the housing.

The sensor housing 220 houses the sensor 235. The sensor 235, for instance, measures the concentration of volatile sulfur chemicals (VSCs), such as hydrogen sulfide (H₂S), in the breath sample. The breath analysis device 100 may include multiple sensors, such as a VSC sensor to determine the concentration of H₂S inside the user's mouth and assess the quality of the user's breath, a humidity sensor to determine the level of hydration of the user, and a pressure sensor to determine the pressure of the sample being taken by the breath analysis device 100.

The sensor housing 220 includes one or more sensor inlet holes 225. The sensor inlet holes 225 may be 0.5 mm to 5 mm in diameter, and the sensor housing 220 may include 1 to 20 sensor inlet holes 225. In some embodiments, the sensor inlet holes 225 are smaller than the outlet holes 215. The sensor inlet holes 225 may be positioned so that the sensor inlet holes 225 do not face the mouthpiece 210. Alternatively, the sensor inlet holes 225 may be located all around the sensor housing 220. The size and number of sensor inlet holes 225 may vary the resistance of a sample entering the sensor housing and thereby passing over the sensor 235.

The housing 205 further includes one or more sensor outlet holes 230. The sensor outlet holes 230 provide an exit path from the housing 205 for the breath sample that entered the sensor housing 220 through the sensor inlet holes 225. The sensor outlet holes 230 may be 0.5 mm to 5 mm in diameter, and the housing 205 may include 1 to 20 sensor outlet holes 230. In some embodiments, the size of the sensor outlet holes 230 is larger than the size of the sensor inlet holes 225. The size of the sensor outlet holes 330 may be substantially equal to the size of the outlet holes 215.

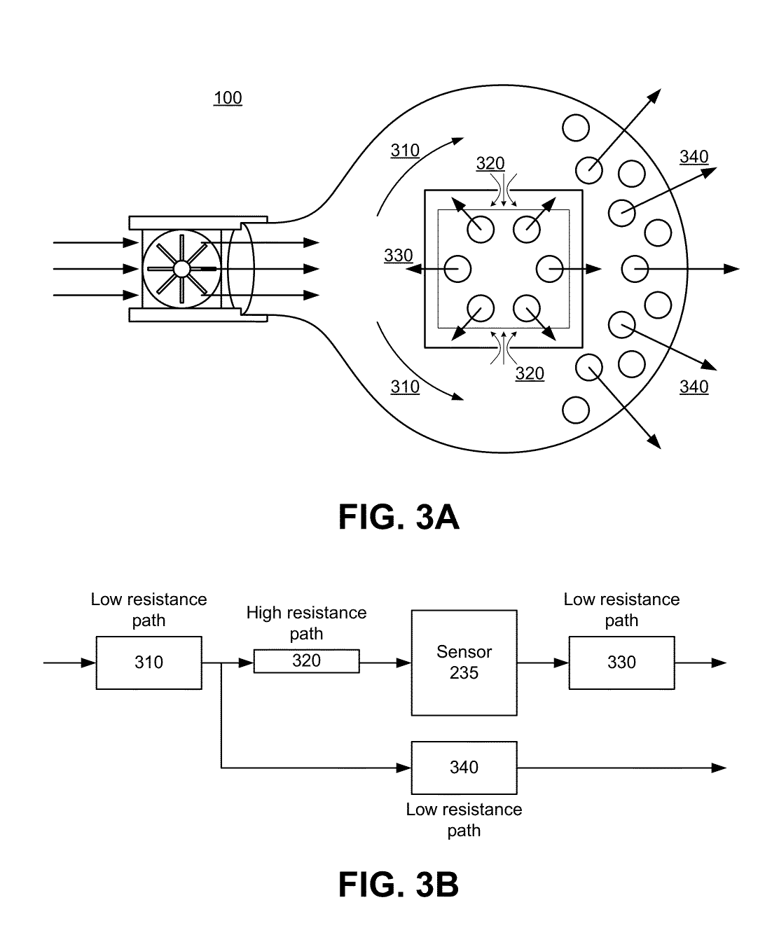

FIG. 3A illustrates a sample breath flow inside the breath analysis device 100, and FIG. 3B illustrates a box diagram of the sample breath flow inside the breath analysis device 100, according to one embodiment. The breath sample enters the breath analysis device 210 via the mouthpiece 210. The breath sample travels through the housing 205 and enters the sensor housing 220 by following based on air paths created by the resistance of the various outlet holes 215, sensor inlet holes 225, and sensor outlet holes 230. The housing 205 forms a first low resistance path 310 for the breath sample to flow through. The breath sample can then either enter the sensor housing 230 through the sensor inlet holes 225 or exit the housing 205 through the outlet holes 215. A high resistance path 320 is provided to the sensor housing 220 while a low resistance path 340 is provided to exit the housing 205. Since the resistance provided by the number and size of sensor inlet holes 225 is higher than the number and size of outlet holes 215, the sensor inlet holes 225 form the high resistance path 320 for the breath sample to flow through, and the outlet holes 225 form the low resistance path 340 for the breath sample to flow through. As a result, a larger amount of the breath sample travels through the housing 205 and exits the housing 205 via the outlet holes 215 relative to the portion of the breath sample that passes into the sensor housing 220.

A smaller amount of breath sample enters the sensor housing 220 via the sensor inlet hole 225 through the high resistance path 320. The sample is analyzed by the sensor 235, and exits the breath analysis device 100 via the sensor outlet holes 230. Since the resistance provided by the number and size of sensor outlet holes 230 is larger than the resistance provided by the number and size, the sensor outlet holes form a low resistance path 330 for the breath sample that entered the sensor housing to exit the breath analysis device 100. As a result, while there is a high resistance path to enter the sensor housing, the low-resistance path reduces the pressure over the sensor 235 relative to the pressure at which the sample passes into the sensor housing.

In some embodiments, since the sensor outlet holes 330 form a low resistance path, the inside of the sensor housing, where the sensor 235 is located, is kept at a relatively constant pressure during the breath sampling. This may further improve the accuracy of the measurement performed by the sensor 235.

In some embodiments, the breath analysis device includes a pump to draw air into the sensor. In this embodiment, the pump may draw a portion of the breath sample after a specified delay and any initial breath sample is discarded.

### Breath Quality Testing

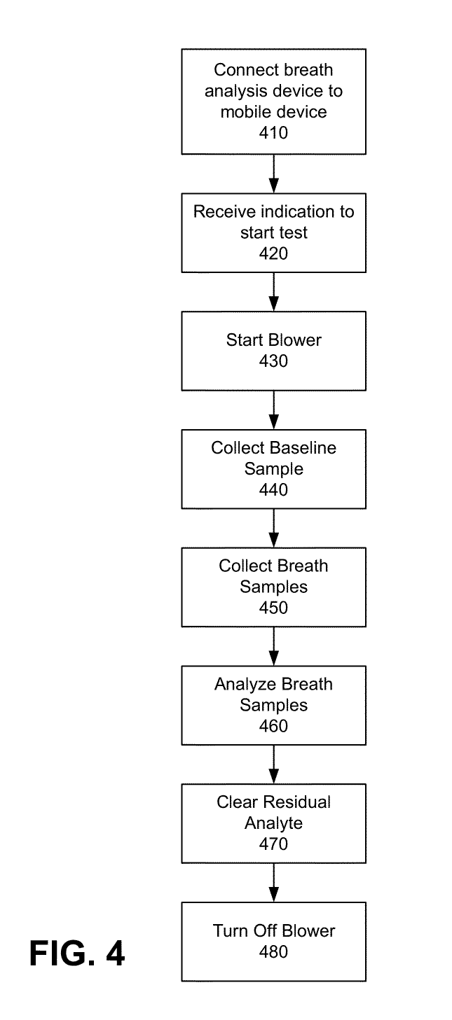

FIG. 4 illustrates a flow diagram of a process for testing the breath quality of a user, according to one embodiment. The breath analysis device 100 is connected 410 to a mobile device 120. For instance, the breath analysis device 100 may be wirelessly connected to a mobile device. In some embodiments, the breath analysis device 100 and the mobile device 120 may synchronize data such as calibration data for the breath analysis sensor of the breath analysis device 100. An indication is received 420 to start the breath quality test. For instance, a user 110 may press a button in the mobile device 120 to which the breath analysis device 100 is connected. The mobile device 120 transmits a signal to the breath analysis device 100 to start the testing of the breath quality of the user 110. The blower 240 of the breath analysis device 100 is turned on 430. The breath analysis device 100 collects 440 a baseline sample. For instance, the breath analysis device 100 collects an air sample before the breath analysis device is placed inside the mouth of the user 110. In some embodiments, the mobile device may indicate the user that a baseline sample is being taken and not to put the mouthpiece 210 of the breath analysis device 100 inside the user's mouth.

After the baseline sample has been taken, the user is instructed to place the mouthpiece 210 of the breath analysis device 100 inside the user's mouth. The breath analysis device 100 detects whether a breath sample is present. In some embodiments, the breath analysis device 100 detects the presence of a breath sample by detecting a change in the humidity of the sample being taken. For instance, if the relative humidity of the sample being take increases above a threshold value, the breath analysis device may determine that a breath sample is being taken instead of a baseline air sample. In some embodiments, if the breath analysis device 100 detects a breath sample, before the breath analysis device has finished collecting the baseline sample, the breath analysis device 100 may cancel the breath quality test. In this embodiment, the breath analysis device 100 may send a signal to the mobile device 120, and the mobile device 120 may display an error message to the user 110.

If a baseline sample has been collected, and the breath analysis does not detect within a threshold time period (e.g., 30 seconds), the blower 240 of the breath analysis device 100 is turned off 480 and the breath quality test is timed out. In some embodiments, the mobile device 120 may display an indication that a breath sample has not been detected, and a countdown of the remaining time before the breath quality test is timed out.

If a baseline sample has been collected, and a breath sample is detected before the breath quality test times out, the breath sample is collected 450. If enough breath samples have been collected (e.g., breath samples have been collected for 25 seconds, or a saturation point in the sensor output has been detected), the breath sample is analyzed 460, the sensor chamber is cleared 470 of residual analyte, and the blower is turned off 480. If not enough breath samples have been collected and the breath analysis device 100 determines that a breath sample is still present, further breath samples are collected 450. Otherwise, if not enough breath samples have been collected, and the breath analysis device determines that a breath sample is not present, the breath quality test is canceled, the sensor chamber is cleared 470 of residual analyte, and the blower is turned off 480.

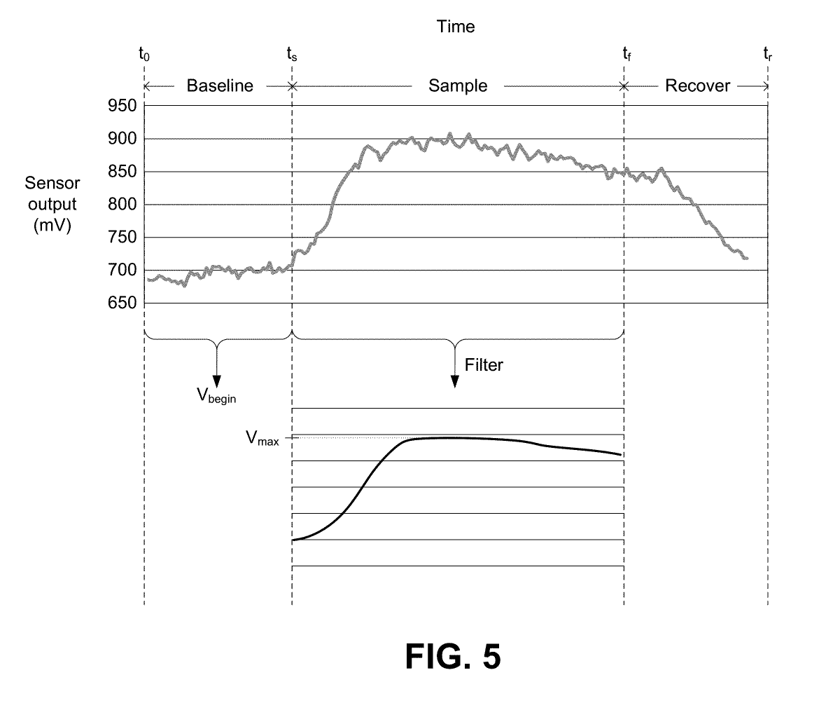

FIG. 5 illustrates a graph of the output of the sensor 235 during an example breath quality test. At time t₀, the blower 240 is turned on and a baseline sample is collected, at time tₛ, a breath sample is detected, at time t_{f}, the collection of the breath sample is completed, and at time tᵣ, the sensor chamber has been cleared of residual analyte. The sensor 235 produces a signal based on the samples taken. For instance, the sensor 235 produces a voltage signal based on the concentration of analytes present in the samples collected. Based on the signal generated by the sensor 235 for the baseline samples, a value V_{begin} is determined. The signal generated between tₛ and t_{f} is filtered. For instance, a fifth order Butterworth low pass filter may be used to filter the signal generated between tₛ and t_{f}. Based on the filtered signal, a value Vₘₐₓ is determined. For instance, a peak value of the filtered signal is determined. In another embodiment, Vₘₐₓ is determined as the peak value of the raw sensor output (i.e., the signal generated by the sensor without being filtered).

During the time interval between t_{f} and tᵣ, the sensor chamber is cleared of residual analyte. In one embodiment, the sensor chamber is cleared of residual analyte by keeping the blower 240 on for a set amount of time after a breath sample is not detected. In other embodiments, the blower 240 is kept on until the output of sensor 235 is below a set value (e.g., below V_{begin}).

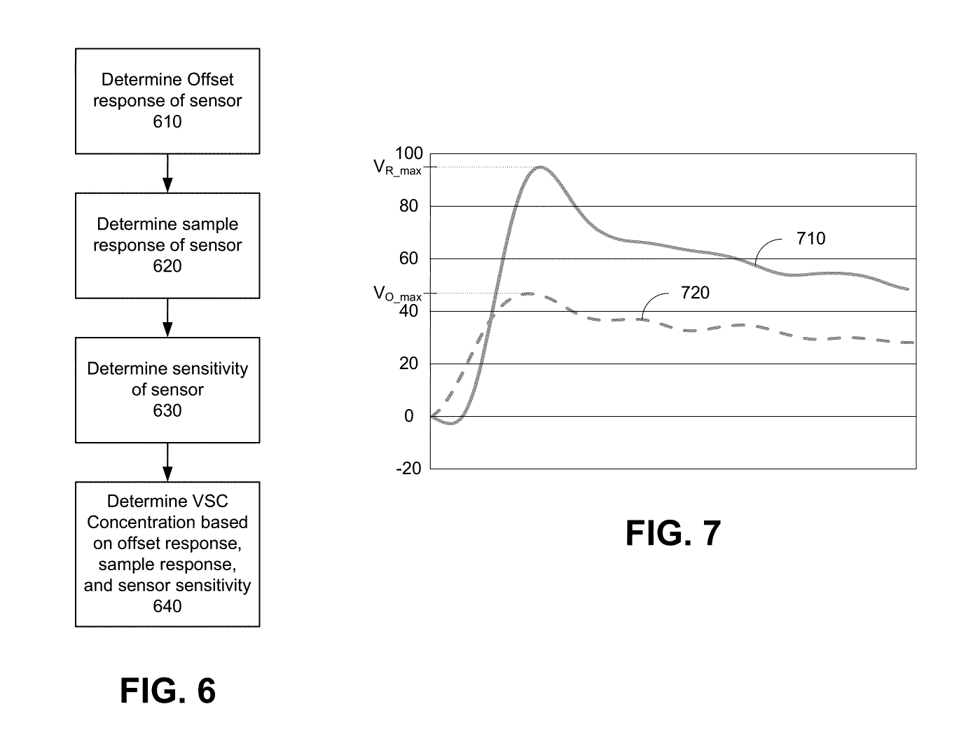

FIG. 6 illustrates a flow diagram of a process for determining a VSC concentration in a user's breath, according to one embodiment. After the baseline sample and the breath sample have been taken, and an output for the baseline sample and the breath sample have been obtained from the sensor 235 a VSC concentration is determined. That is, the output of the sensor (e.g., an output voltage of the sensor in response to a reaction with an analyte present in the samples) is converted into a quantitative measurement of the concentration of the analyte in the sample. To determine the quantitative measurement of the concentration of the analyte in the sample, an offset response is determined for the sensor 235 of a breath analysis device 100. The offset response is the output pattern of the sensor 235 to a sample without the analytes of interest (e.g., free of VSC) but otherwise having similar properties as a breath sample (e.g., high relative humidity). In some embodiments, the offset response is retrieved from a storage device of the client device 120 or the breath analysis device 100. In some embodiments an offset response is determined for each sensor 235 (e.g., by a manufacturer of the sensor 235) and provided to the client device 120 or the breath analysis device 100 to be stored in the storage device. In other embodiments, a single offset response is determined for sensors manufactured in the same lot. For instance, one or more representative sensors may be selected from breath analysis sensors manufactured in the same lot, and an offset response is determined for the selected representative sensors and associated to every sensor in the lot. In some embodiments, representative sensors may be tested periodically to determine a variation in the offset response and the offset responses of the sensors may be updated accordingly.

The offset response is determined by exposing the sensor 235 to a heated water bath (e.g., a sample with a high relative humidity (RH) and with a temperature of 37° C.). FIG. 7 illustrates an offset response 720 after being adjusted for a baseline response (V_{begin}) of a sensor exposed to a sample with a high RH and with a temperature of 37° C. Based on the response of the sensor, an offset response is determined. For instance, a peak value (V_{O}_{_}ₘₐₓ) may be determined based on the filtered response of the sensor 235 to the sample with high RH and a temperature of 37° C. The determined offset response is stored and retrieved when performing a breath quality test.

Returning to FIG. 6, a sample response of the sensor 235 is determined 620. The sample response is the output of the sensor 235 to a breath sample. In some embodiments, the sample response is the output of the sensor 235 to a breath sample after being filtered by a low pass filter. FIG. 7 illustrates a sample response 710 after being adjusted for a baseline response (V_{begin}). A peak value (V_{R}_{_}ₘₐₓ) is determined based on the filtered response of the sensor 235 to a breath sample.

The sensitivity of the sensor 235 is determined 630. The sensitivity of the sensor 235 is the relationship between the output of the sensor 235 to a specific sample and the concentration of the analyte of interest (e.g., VSC) in the specific sample. In some embodiments, the sensitivity is obtained by exposing the sensor 235 to different concentrations of dry H₂S gas (e.g., exposing the sensor 235 to dry H₂S gas with a concentration of 250 ppb). In some embodiments, the sensitivity of the sensor is retrieved from a storage device of the client device 120 or the breath analysis device 100. In some embodiments the sensitivity of the sensor 235 is measured for each breath analysis device 100 (e.g., by a manufacturer of the sensor 235) and provided to the client device 120 or the breath analysis device 100 to be stored in the storage device. In other embodiments, a single sensitivity is measured for sensors manufactured in the same lot. For instance, one or more representative sensors may be selected from sensors manufactured in the same lot, and an sensitivity is measured for the selected representative sensors and associated to every sensor in the lot. In some embodiments, representative sensors may be tested periodically to determine a variation in the sensitivity and the sensitivity of the sensors may be updated accordingly.

Based on the determined offset response, the determined sample response, and the determined sensitivity, the VSC concentration in the breath sample is determined 640. In one example, the VSC concentration is determined as:

$${C}_{V S C} = \frac{{V}_{R\_max} - {V}_{O\_max}}{S}$$

where C_{VSC} is the VSC concentration in the breath sample and S is the sensitivity of the sensor to H₂S. As such, the VSC measurement determined by sensor 235 is corrected for the high relative humidity that is present inside the mouth of a user.

In addition to measuring certain analytes (e.g., VSC), the breath analysis device 100 determines the relative humidity of the breath sample to determine the hydration level of the user. Dehydration is also related to poor breath quality and thus determining a hydration level of the user in addition to the concentration of the aforementioned analytes aids the breath analysis device 100 to determine the root cause of the poor quality of the user's breath.

Breath samples have a high relative humidity and thus, the time for a humidity sensor to produce a saturated response based on the humidity of the sample may be longer than the time for the sensor 235 to produce a saturated response based on the concentration of the analytes in the sample. To shorten the time to obtain a relative humidity measurement, the output of the sensor 235 may be extrapolated. In some embodiments, the measurements are extrapolated using a predefined model. For instance, the humidity measurements are extrapolated to determine a predicted saturation value based on a model fitted to the measurement values previously determined by the sensor 235. In one embodiment, a Gompertz 3P function is used to model the humidity measurements and predict a saturation value of the humidity measurements. The Gompertz 3P function may look as:

y(t)=ae^{-be}^{^{−ct}}

where y(t) is the output of the model, a, b, and c are fitting parameters, and t time.

### User Interface for Breath Analysis System

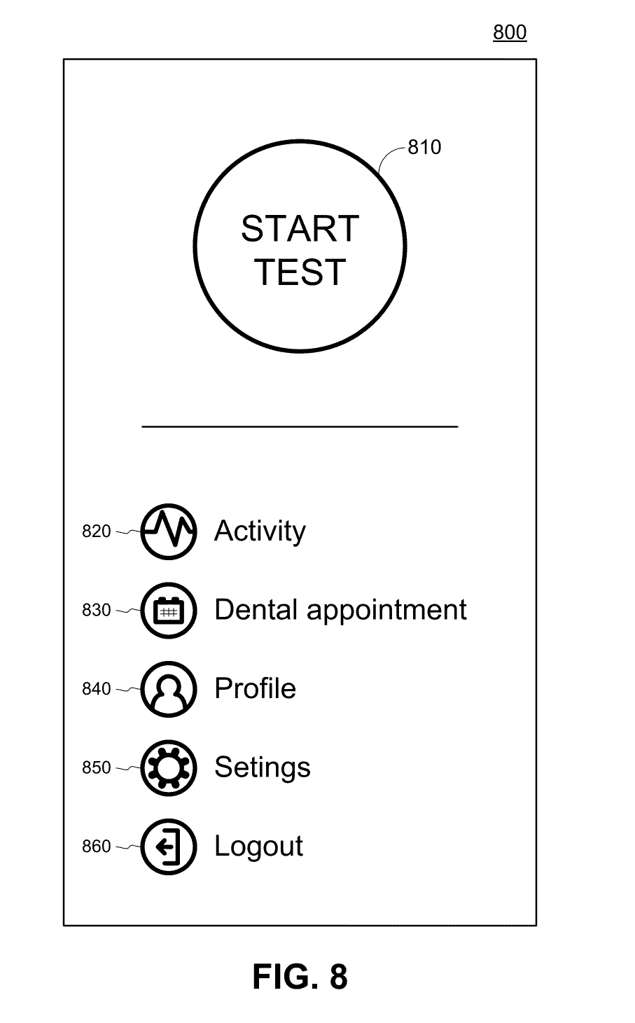

FIG. 8 illustrates a user interface 800 for controlling the breath analysis device, according to one embodiment. The user interface 800 includes a button 810 to start a breath test. Upon receiving an indication to start a breath test, the client device 120 initializes the breath analysis device 100 (e.g., connects to the breath analysis device 100 via Bluetooth) and shows the user 110 instructions on the steps to perform during the analysis of the user's breath. A user interface for showing instruction on the steps to perform during the analysis of the user's breath is shown in conjunction with FIG. 9.

The user interface 800 may also include a button 820 to display data from previous test performed by the breath analysis device 120, a button 830 to interact with a third party system (e.g., to schedule a dental appointment with the user's dental care provider), a button 840 to see and/or modify the user's profile information, a button 850 to see and modify the settings of the breath analysis device, and a button 860 to logout the user's account from the client device 120. Some embodiments may include additional or fewer buttons or user interface elements than the ones illustrated in FIG. 8.

FIG. 9 illustrates a user interface 900 for providing instructions to a user during the analysis of the user's breath, according to one embodiment. The user interface 900 shows the user 110 instructions 910 on the steps to perform during the analysis of the user's breath. For instance, the user interface 900 of FIG. 9 is instructing the user to place the breath analysis device 100 inside the user's mouth for 5 seconds. The instructions may also instruct the user not to exhale through the mouth during the breath measurement. Additionally, the user interface 900 includes a countdown of the number of seconds left for the analysis. The user interface 900 of FIG. 9 instructs the user to keep the breath analysis device 100 inside the user's mouth for 2 more seconds. In some embodiments, the countdown starts when the breath analysis device 100 detects that the user has placed the breath analysis device 100 inside the user's mouth.

The breath analysis device 100 may provide information to the client device 120 while a breath sample is being obtained. This information may indicate, for example, fluctuations in the breath sample, sensor readings, or other information. The client device 120 in some embodiments provides additional instructions to the user based on this information, for example to stop blowing into the breath analysis device 100, or not to block the outlet holes 115. In some embodiments, the breath analysis device 100 may detect whether a user is exhaling through the mouth (e.g., by detecting an increase in pressure of the breath sample) and discard the measurement results. If the pressure of the breath sample is higher than a threshold value, the breath analysis device 100 may stop the analysis and display a visual cue to the user to restart the analysis and to avoid exhaling into the breath analysis device.

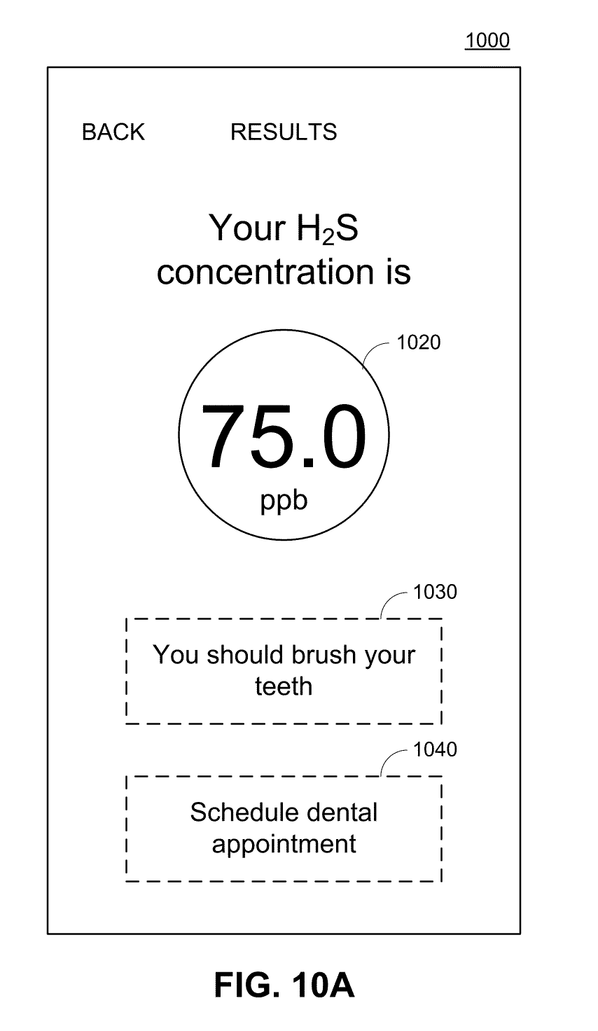

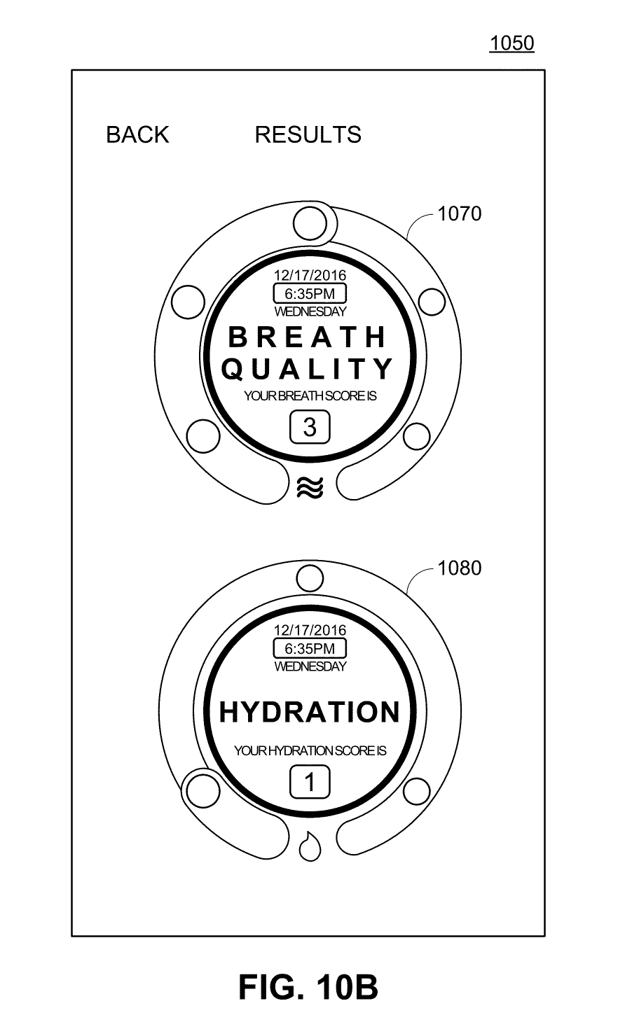

FIG. 10A illustrates a user interface 1000 for providing the analysis results, according to one embodiment. The user interface 1000 includes a graphical element 1020 that shows the H₂S concentration inside of the user's mouth. In this example user interface, the H₂S concentration is 75.0 ppb. The user interface 1000 may display additional information, such as the humidity level inside the user's mouth.

In some embodiments, client device 120 determines suggestions for the user based on the measurement results. The user interface 1000 displays the suggestions 1030 to the user based on the results of the analysis. For instance, if the user has a H₂S concentration that is greater than a threshold limit, the user interface 1000 may suggest the user to brush their teeth. In some embodiments, the suggestion is an interface to perform the suggested task. For instance, selecting graphical element 1040 may display an interface to schedule an appointment with the user's dental care provider. In another example, if the humidity inside the user's mouth is lower than a threshold, the user interface 1000 may suggest the user to drink more water.

In some embodiments, the client device 120 determines suggestions based on multiple breath analysis results. For instance, the client device 120 determines suggestions based on results from analyses performed in a set time period (e.g., past 7 days). If the results of the breath analyses indicate that the user had an H₂S concentration higher than a threshold value for more than a threshold number of days in the set time period, the user interface 1000 may suggest the user to visit a dental hygienist.

In some embodiments, the user may be able to provide the client device with information regarding the user's diet (e.g., the food the user consumed each day, or the amount of water consumed by the user each day), and the client device 120 may provide suggestions on how to improve the user's oral quality based on the information provided by the user.

FIG. 10B illustrates a user interface 1050 for providing the analysis results, according to one embodiment. The user interface 1050 includes a graphical element 1070 that shows a breath quality score, and a graphical element 1080 that shows a hydration score 1080. The mobile device determines a score based on the VSC concentration of the breath sample determined by the sensor 235. Scores may be associated with VSC concentration ranges. For instance, a VSC concentration greater than 500 ppb is associated with a score of 1, a VSC concentration between 300 ppb and 500 ppb is associated with a score of 2, a VSC concentration between 140 ppb and 300 ppb is associated with a score of 3, a VSC concentration between 50 ppb and 140 ppb is associated with a score of 4, and a VSC concentration lower than 50 ppb is associated with a score of 5. Similarly, a hydration score is determined by the mobile device based on the relative humidity in the breath sample as determined by the sensor 235. In the exemplary user interface 1050, a breath quality score of 3 out of 5, and a hydration score of 1 out of 3 is determined for the user. In some embodiments, an oral quality score is determined based on multiple parameters. For instance, a single oral quality score may be determined based on both the VSC concentration and the relative humidity of the breath sample.

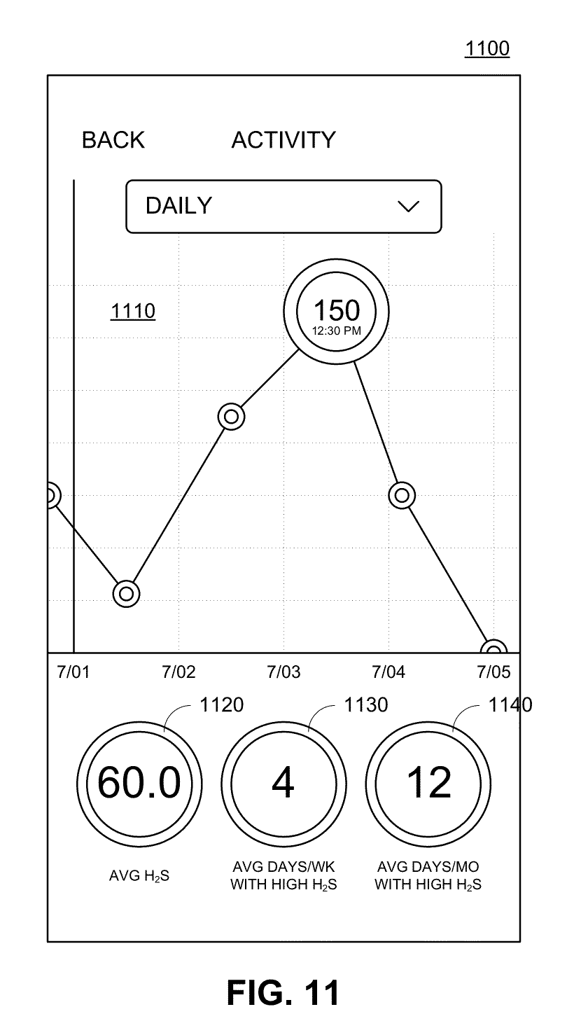

FIG. 11 illustrates a user interface 1100 to display data from previous tests performed by the breath analysis device, according to one embodiment. The user interface 1100 includes a graph 1110 that shows the results of previous analysis as a function of time. The graph 1110 may also indicate the time and the value of the worst result in the time range plotted by the graph. The user interface 1100 shows additional information such as an average H₂S concentration, an average number of days per week with an H₂S concentration higher than a threshold value, and an average number of days per month with an H₂S concentration higher than the threshold value. For instance, the user interface 1100 includes a user interface element 1120 that shows the average H₂S concentration in a given time range, a user interface element 1130 that shows the average number of days per week with an H₂S concentration higher than a threshold value, and a user interface element 1140 that shows the average number of days per month with an H₂S concentration higher than the threshold value.

In some embodiments, the client device may connect with a third party system, such as the user's dental care provider, and provide the third party system with the results of the analysis. Using the data provided by the client device 120, a dentist may be able to give the user 100 advice on how to improve the user's oral hygiene, or a dental health insurance provider may use the results provided by the client device 120 to reduce the user's dental insurance premiums.

### Additional Configuration Considerations

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs)).

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

What is claimed is:

1. A breath quality analysis device comprising:
a housing having an inlet opening, one or more outlet openings, and an inner cavity, the inner cavity defining a path between the inlet opening and the one or more outlet openings;
a sensor disposed inside the inner cavity of the housing; and
a mouthpiece, the mouthpiece coupled to the inlet opening of the housing, the mouthpiece comprising a blower configured to draw air into the inner cavity of the housing.

2. The breath quality analysis device of claim 1, further comprising:
a wireless transceiver, the wireless transceiver configured to wirelessly communicate with a mobile handheld device.

3. The breath quality analysis device of claim 2, wherein the wireless transceiver transmits messages using Bluetooth.

3. The breath quality analysis device of claim 3, wherein the wireless transceiver transmits messages using Bluetooth Low Energy (BLE).

4. The breath quality analysis device of claim 1, wherein the mouthpiece further comprises:
one or more grooves disposed in an outer surface of the mouthpiece, wherein the grooves are shaped for a contour of a person's teeth.

5. The breath quality analysis device of claim 1, wherein the mouthpiece is detachably coupled to the housing.

6. The breath quality analysis device of claim 1, wherein the mouthpiece is non-detachably coupled to the housing.

7. The breath quality analysis device of claim 1, wherein the blower comprises a rotor having multiple blades.

8. The breath quality analysis device of claim 7, wherein the blades are arranged vertically with respect to an axis of rotation of the rotor.

9. The breath quality analysis device of claim 7, wherein the blades are slanted with respect to an axis of rotation of the rotor.

10. The breath quality analysis device of claim 1, wherein the inner cavity of the housing comprises:
a sensor housing for housing the sensor;
one or more sensor housing inlet holes, the one or more sensor housing inlet holes configured to allow a portion of the air drawn into the inner cavity of the housing by the blower to enter the sensor housing;
a first set of paths from the one or more inlet openings to the one or more sensor housing inlet holes; and
a second set of paths from the one or more inlet openings to a first subset of outlet opening of the one or more outlet opening, wherein the second set of paths does not pass though the sensor housing.

11. The breath quality analysis device of claim 10, further comprising:
a third set of paths from the one or more sensor housing inlet holes to a second subset of outlet openings of the one or more outlet openings.

12. The breath quality analysis device of claim 11, wherein the third set of paths is disposed inside the sensor housing.

13. The breath quality analysis device of claim 10, wherein the sensor housing inlet holes have a smaller diameter than the outlet openings.

14. The breath quality analysis device of claim 1, wherein the sensor is configured to detect the presence volatile sulfur compounds (VSC) in an air sample.

15. The breath quality analysis device of claim 1, wherein the sensor is configured to detect humidity in an air sample.

16. A method for determining an oral quality of a user, comprising:
receiving a plurality of volatile sulfur compound (VSC) and relative humidity (RH) measurements from a breath quality analysis device;
determining a baseline response and a sample response based on the plurality of VSC measurements;
determining a baseline shift based on the baseline response;
filtering the sample response using a low pass filter to generate a filtered sample response;
determining a peak sample response based on the filtered sample response; and
determining the oral quality of the user based on the baseline shift and the peak sample response.

17. The method of claim 16, wherein an end of the baseline response and a start of the sample response within the plurality of VSC measurements is determined based on a change in the received RH measurements.

18. The method of claim 16, further comprising:
determining a saturation value of the RH measurements comprising:
fitting the RH measurements to a growth model, and
determining a saturation value of the fitted growth model.

19. The method of claim 18, wherein the low pass filter is a Butterworth low pass filter, and the growth model is a Gompertz function.
